(19) United States
(12) Patent Application Publication
(10) Pub. No.: US 2010/0160947 A1
(43) Pub. Date: Jun. 24, 2010

(54) SYSTEMS AND METHODS FOR DILATION AND DISSECTION OF TISSUES

(75) Inventors: Ephraim Akyuz, Providence, UT (US); Kabir Gambhir, San Diego, CA (US); Corbet W. Stone, San Diego, CA (US)

Correspondence Address: IMDS, INC., 124 SOUTH 600 WEST, LOGAN, UT, 84321, US

(73) Assignee: IMDS, INC., Logan, UT (US)

(21) Appl. No.: 12/640,413

(22) Filed: Dec. 17, 2009

Related U.S. Application Data
(60) Provisional application No. 61/138,629, filed on Dec. 18, 2008.
(60) Provisional application No. 61/166,069, filed on Apr. 2, 2009.

(51) Int. Cl. A61M 29/02; A61M 29/00; A61B 5/00

(52) U.S. Cl. 606/192; 606/198; 600/554

(57) ABSTRACT

A minimally invasive dilation device includes a stylus, a plurality of rigid arms radially arrayed about the stylus, and a dilating member positioned between the stylus and the arms. An outer flexible sleeve may be circumferentially secured to the arms, lying within or without the plurality of arms. An inner mesh may surround the stylus and dilating member. The device may be introduced into tissue toward a targeted area, while in a closed configuration. The dilating member may be a balloon, wherein upon inflation of the balloon, the arms are pushed radially outward, expanding the device and dilating the surrounding tissue. A cannula may be inserted inside the plurality of arms to keep the arms in an open configuration, and the stylus, balloon and inner mesh may be withdrawn, providing an open passageway through the device to the targeted area. The device may be used with a neural monitoring system.

### CROSS-REFERENCE TO RELATED APPLICATIONS

This application claims the benefit of the following:

pending U.S. Provisional Patent Application No. 61/138,629, filed Dec. 18, 2008, which carries Applicants' docket no. INS-7 PROV, and is entitled SYSTEMS AND METHODS FOR DILATION AND DISSECTION OF TISSUES DURING LATERAL SPINE ACCESS SURGERY; and

pending U.S. Provisional Patent Application No. 61/166,069, filed Apr. 2, 2009, which carries Applicants' docket no. MLI-75 PROV, and is entitled SYSTEM AND METHOD FOR DILATION AND DISSECTION OF TISSUES.

The above-identified documents are incorporated herein by reference.

### BACKGROUND OF THE INVENTION

1. The Field of the Invention

The invention relates to orthopaedics, and more particularly, to providing access to a surgical site in the body through the use of an expandable minimally invasive dilation device.

2. The Relevant Technology

Many spinal orthopaedic procedures including discectomy, implantation of motion preservation devices, total disc replacement, and implantation of interbody devices require unimpeded access to a targeted portion of the spinal column. Providing access to the targeted area may require forming a passageway through muscles, fascia and other tissues. Current surgical access systems utilize a series of sequential dilators, or a mechanical retractor system with at least one dilating cannula.

There are several disadvantages associated with sequential dilators. Sequential dilator systems can shear the tissues through which they are advanced. These tissues can include muscle, nerves, blood vessels, and organs. In addition, the tissues at the distal end of the dilators can be crushed against bone or other soft tissues rather than properly separated. As multiple dilators are deployed to enlarge a space, the tissues may be repeatedly injured as each dilator is advanced through the same tissues.

Accordingly, there is a need in the art for systems and methods that facilitate access to the spine, while minimizing trauma to surrounding tissues and avoiding time-consuming and unnecessary repetitive steps. Keeping the overall diameter and the number of passes of the cannulas to a minimum may minimize the trauma to the surrounding structures. Such systems and methods can simplify surgical procedures and expedite patient recovery. Ultimately, reducing the invasiveness of the procedure will result in faster recoveries and improved patient outcomes.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1A is a perspective view of a tissue dilation device in a closed configuration and attached to a hub, the device comprising a stylus, a balloon, a plurality of arms surrounding the stylus, an inner mesh, and an outer sheath;

FIG. 1B is a perspective view of the tissue dilation device of FIG. 1A in the closed configuration, with the outer sheath not depicted, and dashed lines representing the inner mesh;

FIG. 2 is a perspective view of the tissue dilation device of FIG. 1A, with the balloon inflated and the device in an expanded configuration, and dashed lines representing the outer sheath;

FIG. 3 is a perspective view of the tissue dilation device of FIG. 1A in the expanded configuration, with the outer sheath not depicted, and dashed lines representing the inner mesh;

FIG. 4A is an enlarged cross-sectional longitudinal view of the distal end of the tissue dilation device of FIG. 1A in the expanded configuration;

FIG. 4B is an enlarged cross-sectional end view of the distal end of the of the tissue dilation device of FIG. 1A in the expanded configuration, taken along line a-a of FIG. 4A;

FIG. 5A is a perspective view of a distal portion of the stylus of FIG. 1A, with dashed lines representing an inner bore;

FIG. 5B is a perspective view of an outer side of one arm of the plurality of arms of FIG. 1A;

FIG. 5C is a perspective view of an inner side of one arm of the plurality of arms of FIG. 1A;

FIG. 5D is an enlarged cross-sectional transverse view of the plurality of arms of FIG. 1A in the closed configuration;

FIG. 6A is a perspective view of the tissue dilation device of FIG. 1A in the closed configuration, with the distal end inserted into a psoas muscle adjacent a vertebra, and the proximal end attached to the hub;

FIG. 6B is a perspective view of the tissue dilation device of FIG. 6A in the open configuration, with the stylus, balloon, and inner mesh withdrawn, and an open passageway extending through the hub, dilation device and psoas muscle;

FIG. 7 is a side view of a curved tissue dilation device in a closed configuration, the device comprising a stylus, two balloons, and a plurality of curved arms radially surrounding the stylus and the balloons, wherein the arms are releasably secured to the proximal end of the stylus, and the arms are releasably secured to one another via lateral engagement features fastened by a plurality of release wires;

FIG. 8 is a top exploded view of the stylus and two arms of the curved tissue dilation device of FIG. 7;

FIG. 9 is a partially exploded enlarged view of the distal end of the stylus, balloon and arms of the curved tissue dilation device of FIG. 7;

FIG. 10 is a perspective view of the tissue dilation device of FIG. 7 in an expanded configuration, with a luer attached to the proximal end of the stylus;

FIG. 11 is a perspective view of the tissue dilation device of FIG. 7 in an expanded configuration, with a cannula partially inserted into the device;

FIG. 12 is a perspective view of a stylized cross-section of a human body, with a curved tissue dilation device in a closed configuration inserted into a psoas muscle, and connected to a targeting system positioned to target a predetermined location along the spine;

FIG. 13 is a perspective view of the body, curved tissue dilation device and targeting system of FIG. 12, with the dilation device in an open configuration and the dilation device and targeting system secured to table mounted clamps, and with a cannula partially inserted into the dilation device;

FIG. 14 is a perspective view of the body, curved tissue dilation device and targeting system of FIG. 12, with the cannula fully inserted into the dilation device; and

FIG. 15 is a perspective view of the body and targeting system of FIG. 12, with an electromyography electrode inserted into the psoas muscle and connected to a neural monitoring system.

### DETAILED DESCRIPTION OF THE PREFERRED EMBODIMENTS

The present invention relates to systems and methods for dilating tissues to provide access to intervertebral space or other targeted areas. Those of skill in the art will recognize that the following description is merely illustrative of the principles of the invention, which may be applied in various ways to provide many different alternative embodiments. This description is made for the purpose of illustrating the general principles of this invention and is not meant to limit the inventive concepts in the appended claims.

The present invention provides access to the spine through the use of a minimally invasive expandable dilation device. The device may be placed within the tissue with a minimal profile, yet has a high expansion ratio, with the result that the expanded device provides an optimally sized passageway allowing access to the targeted spinal area, with minimal impact on surrounding tissues. A single device is advanced into the tissues to be dilated, and expanded from within. Thus additional steps of introducing successive dilators are avoided, along with repetitive damage to the tissues caused by forcing dilator after dilator through the tissues.

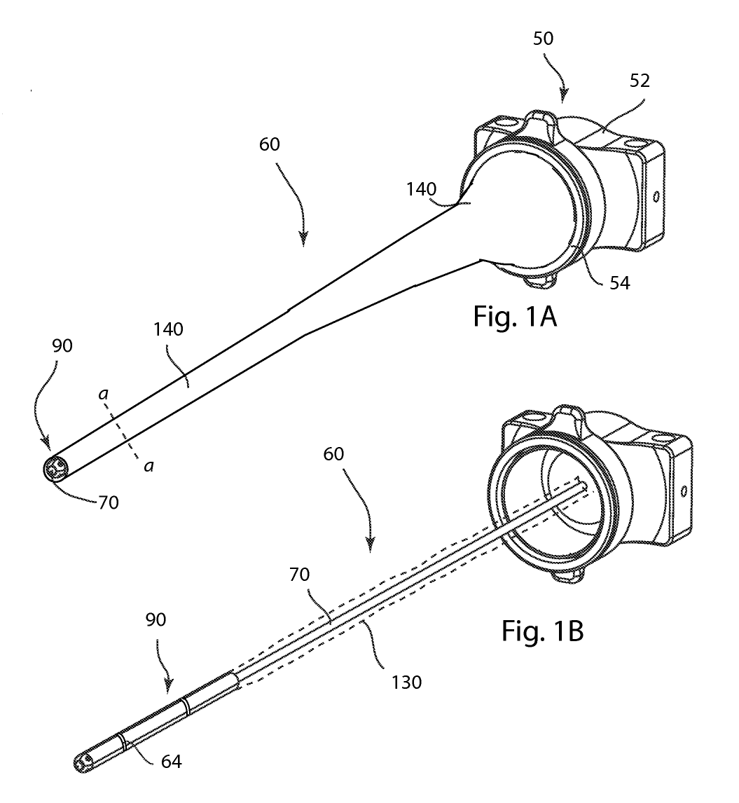

FIGS. 1-6B display views of one embodiment of a dilation device 60. The dilation device 60 comprises an obturator or stylus 70, a plurality of rigid arms 90, a balloon 110, a flexible inner mesh 130, and an optional, flexible outer sleeve or sheath 140. A portion of the dilation device may be introduced into a muscle, and the dilation device expanded from a closed configuration to an open configuration to dissect and separate the muscle fibers and form a passage through the muscle. After expansion, the stylus, balloon and inner mesh may be removed, leaving an open passage through the muscle, through which instruments, implants and other materials may be passed to perform one or more surgical procedures.

Referring to FIGS. 1A and 1B, the dilation device 60 is shown in a closed configuration, partially extending through a hub 50. The hub 50 comprises a hub body 52 and a collet 54, and is attachable to a surgical table mounted support system (not shown), which may provide stability and support to the hub and dilation device during surgical procedures. In FIG. 1A, of the device 60 only the optional outer sleeve 140, and the distal ends of the plurality of arms 90 and the stylus 70 are visible, as the outer sleeve 140 obscures most of the device. The outer sleeve 140 is securely attached to the plurality of arms, and circumferentially securely attached to the collet, forming a barrier around the remainder of the device. FIG. 1B depicts the device without the outer sleeve 140, and with dashed lines representing the inner mesh 130. The balloon 110 is mounted circumferentially on the stylus 70 toward the distal end of the stylus and extends proximally along a portion of the stylus. The plurality of arms 90 may completely surround the balloon in the closed configuration; hence the balloon is not visible in FIGS. 1A and 1B. The inner mesh 130 surrounds the balloon, interposed between the balloon and the plurality of arms, and extending from a distal end of the plurality of arms toward the collet 54. The inner mesh may be attached to the stylus. The maximum outer diameter of the device 60 in the closed configuration, measured normal to the longitudinal axis of the stylus and rigid arms, such as along line a-a, may range from 5 to 15 millimeters.

Device 60 may further comprise one or more retention bands 64 which are placed around the plurality of arms when the device is in the closed configuration, to aid in holding the device closed. The bands may comprise a biocompatible polymer, which may be bio-absorbable, and have a generally circular ring shape. The bands may be heat-shrunk about the closed device. During expansion, as the arms move radial-laterally relative to one another, the force of the moving arms will stretch and ultimately break the band(s). Any of the dilation devices disclosed herein may comprise these retention bands.

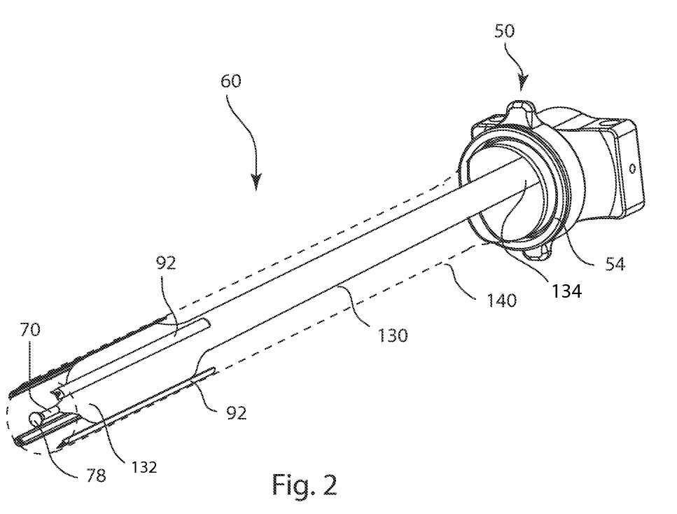

FIG. 2 shows the dilation device 60 in an expanded, or open configuration. The outer sleeve 140 is not depicted but its location is indicated with dashed lines. A distal portion 132 of the inner mesh 130 surrounds the balloon, and in this embodiment the distal end of the mesh is attached to the stylus. A proximal end of the mesh 134 extends through the collet 54, surrounding the stylus.

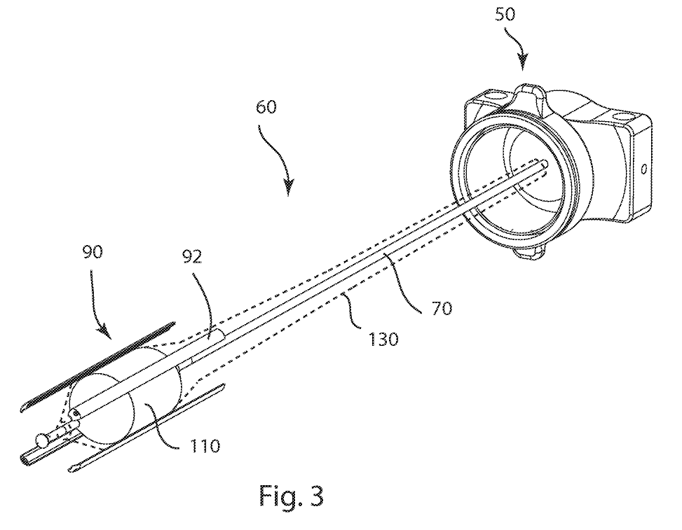

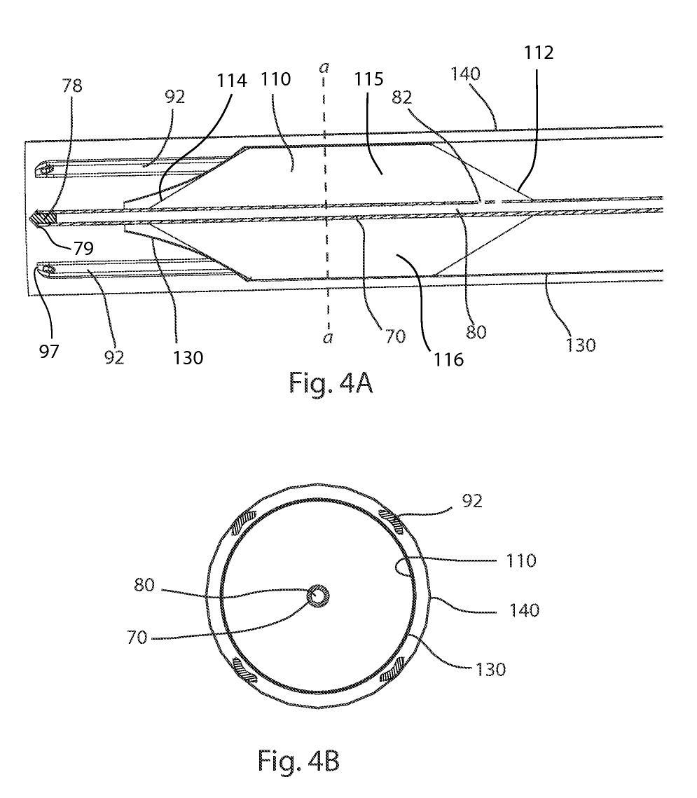

FIG. 3 shows the dilation device 60 in the expanded configuration, without the outer sleeve, and the inner mesh is indicated by dashed lines. FIG. 4A shows an enlarged longitudinal cross-sectional view of a distal portion of the device 60 in the expanded or open configuration. FIG. 4B shows a further enlarged cross sectional view of the distal end of the device 60 in the open configuration, taken along line a-a of FIG. 4A. Each end of the balloon 110 is attached to the stylus 70. To attain the open configuration, fluid is introduced through the stylus into the balloon 110, inflating the balloon. As the balloon 110 is actuated by inflation, it expands radially, and an outer surface of the balloon pushes against the plurality of rigid arms 90, and each individual arm 92 is displaced radially outward and laterally away from the adjacent arms. The inner mesh 130, which surrounds a body of the balloon, also expands or unfolds radially outward, generally conforming to the shape of the balloon where it is adjacent to the balloon. The outer sleeve 140 also expands or unfolds with expansion of the device. After expansion of the device to the open configuration, the stylus 70 and attached balloon 110 and inner mesh 130 may be withdrawn proximally, leaving an open passageway extending from the hub 50 to the distal ends of the arms 92, the open passageway lined by the arms and the outer sleeve. The device 60 comprises a substantially cylindrical shape in both the closed configuration, as seen in FIG. 1A, and the open configuration, as seen in FIG. 2.

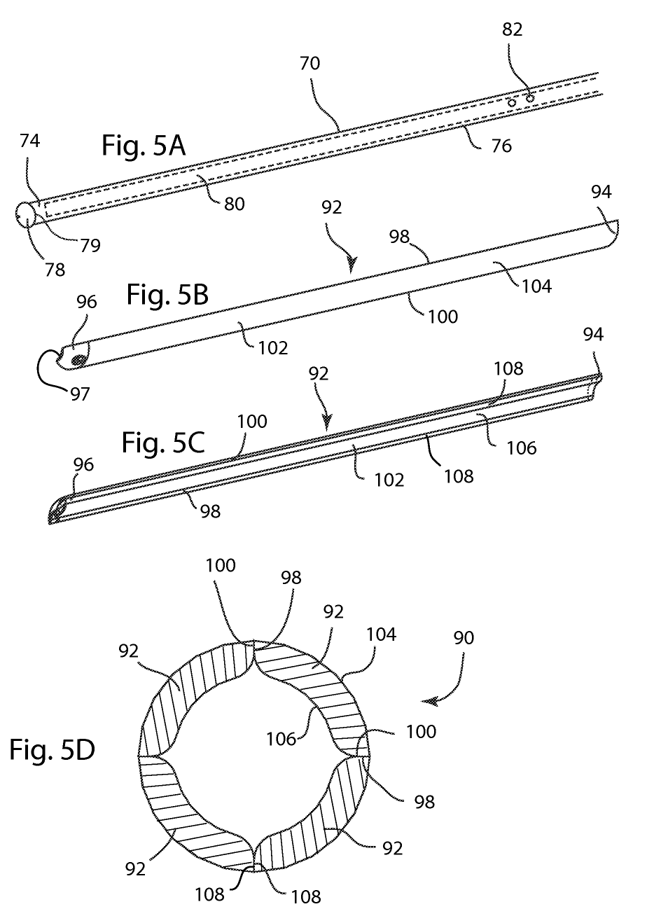

FIGS. 5A through 5D display details of the stylus 70 and arms 92. Referring to FIGS. 3, 4A and 5A, stylus 70 comprises a proximal end 72, distal end 74, and a shaft 76 extending between the proximal and distal ends. The stylus may also be an obturator or a hypotube, comprising stainless steel or another biocompatible metal. A stylus tip 78 is located at the distal end and may be formed integrally with the stylus, or formed separately from the stylus and rigidly secured to the stylus. The tip 78 may be blunt, to separate and push aside muscle fibers with minimal trauma to the fibers during advancement of the stylus into the muscle. In some embodiments, the tip may be conical, pointed and/or comprise a cutting edge. In some embodiments, the stylus distal end may further comprise connecting features which cooperate with complementary connecting features on the rigid arms to place the arms in a predetermined longitudinal alignment with the stylus when the connecting features are engaged with one another. The stylus may be partially hollow, having an inner bore 80 extending from an opening at the proximal end, to or toward the distal end. One or more ports 82 may penetrate from the bore 80 to the outside of the shaft 76, through which fluid may flow to inflate the balloon during dilation. A luer (not shown) may be attached to the proximal end of the stylus, in communication with the bore 80, for introduction of fluids into the stylus bore.

Referring to FIGS. 3 through 5D, the plurality of rigid arms 90 may comprise four or more individual arms 92. Each arm 92 may be identical to each other arm, and comprises a proximal end 94, a distal end 96, a first lateral edge 98 and a second lateral edge 100 opposite the first lateral edge. A shaft 102 extends between the proximal and distal ends, bounded laterally by the lateral edges 98, 100. An outer surface 104 which may be convexly curved covers one side of the arm, while an inner surface 106 which may be concavely curved covers the opposite side. Each entire arm 92 may be curved about its longitudinal axis, such that when the arms are positioned in a closed configuration so that their lateral edges are adjacent one another in contacting alignment as in FIGS. 1B, 5D, and 6A, a closed cylinder is formed. The inner diameter of the closed cylinder is sized to receive the stylus 70 and the uninflated balloon 110. The arms may include holes or other features used in secure attachment of the outer sleeve to the arms via sutures, pins, or other attachment mechanisms. In the embodiment shown in FIGS. 1B-3, the arms extend along only a portion of the stylus. In other embodiments, the arms may be longer, and can extend the entire length of the stylus and/or extend out of the hub 50. Each arm 92 may flare or curve outward at its distal end, which may aid in keeping tissues retained during dilation or expansion.

The arm distal end 96 may comprise an arm connection feature which is shaped to engage with a corresponding stylus connection feature to place the arm in a predetermined longitudinal alignment with the stylus. With reference to FIGS. 4A, 5A and 5B, the arm connection feature may comprise a curved distal edge 97. The stylus connection feature may comprise a segment of an overhanging lip 79. The lip 79 comprises a circular flange on the stylus tip 78, which may project outward from the stylus tip. When an arm curved distal end 97 is positioned in abutment with a correspondingly curved segment of the lip 79 such that the entire curved distal end is in contact with the lip segment, the arm is placed in a predetermined longitudinal alignment with the stylus.

The arm lateral edges 98, 102 may comprise complementary engagement features which cooperate with the engagement features on adjacent arms to place the arms in contacting longitudinal alignment with one another along their first and second lateral edges when the arms are in the closed configuration. In one embodiment, the engagement features may comprise planar portions wherein the first lateral edge comprises a planar surface 108 which engages a complementary planar surface on the adjacent second lateral edge. In another embodiment, the engagement features comprise tongue-in-groove features wherein the first lateral edge comprises a tongue while the second lateral edge comprises a groove shaped to receive the tongue. In yet another embodiment, the engagement features may comprise alternating edge extensions with bores shaped to receive a longitudinal member such as a wire or suture, so that the edges may be temporarily laced together. In the closed configuration the longitudinal member extends through the bores and the arms are retained in contacting longitudinal alignment; when the longitudinal member is removed the arms are free to disengage and move apart from one another.

The arms may be at least partially radiolucent, so as not to compromise visualization of procedures during use of the device with fluoroscopy. Alternatively, the arms may be at least partially radiopaque, to assist with positioning and location of the system under fluoroscopy. The arms may comprise metals such as aluminum, stainless steel, titanium, and other biocompatible metals. The arms may also comprise high density plastics such as Delrin, Radel, Udel, poly ether ether ketone (PEEK), polycarbonate, and acrylonitrile butadiene styrene (ABS), among others. Barium sulphate may be added to constituent plastic materials to provide increased radiopacity.

With reference to FIG. 4A, the balloon 110 comprises a proximal end 112, a distal end 114, and a substantially cylindrical balloon body 116 extending therebetween. At the proximal 112 and distal 114 ends, the balloon is circumferentially attached to the stylus through adhesives or other bonding methods such that when fluids are introduced into a balloon lumen 115, the fluids cannot escape at the points of attachment to the stylus 70. As fluid is introduced into the balloon 110 from the stylus 70 through the ports 82, the balloon may inflate proximally to distally. As the proximal end of the balloon inflates, the arms 92 may be pushed slightly distally, then radially outward as the inflation continues distally. The proximal ends 94 of the arms may be pushed radially outward before, or at the same time, as the distal ends 96 of the arms 92.

The balloon may be opaque or translucent, and the balloon may be compliant or non-compliant. A compliant balloon may allow for an even distribution of force on the rigid arms and ultimately the surrounding tissue. A non-compliant balloon may allow for an uneven distribution of force and as such may be well suited for dissection of tissues. The shape of the balloon may be optimized to best suit the physiology and tissue it will dissect. For example, a round balloon may produce uniform force distribution and create a localized open space. An elongated balloon may produce distal expansion to create space at the distal end of the device. The balloon may comprise polyethylene, polyethylene terephthalate (PET), polytetrafluoroethylene (PTFE), nylon, Dacron, polyurethane, or other compliant or non-compliant polymers.

The inner mesh 130 may be fixed to the stylus at a location distal to the distal end of the balloon, extending to or toward the proximal end of the stylus. The inner mesh 130 is generally tubular and flexible, able to conform to the shape of the balloon, and may be permeable or non-permeable. The inner mesh may be of an indeterminate shape or a pre-formed shape. The mesh may comprise polypropylene, polyethylene (PE), polyethylene terephthalate (PET), poly ether ether ketone (PEEK), nylon, ultra-high molecular weight polyethylene (UHMWPE), or any other biocompatible polymer, or a combination thereof. In some embodiments, the inner mesh may be formed such that as a portion of the inner mesh is expanded by the balloon or dilation member, the length of the inner mesh is foreshortened.

In some embodiments, the dilation device 60 may further comprise an outer mesh or sheath 140 which may circumferentially surround the rigid arms and stylus, to further retain and protect bodily tissues during dilation. In other embodiments, the outer sheath may be positioned inside the arms, but outside of and circumferentially surrounding the inner mesh, balloon and stylus. The outer sheath may prevent pinching of tissues and/or migration of tissues between the rigid arms during the dilation process. The outer sheath is securely attached to the arms, whether inside or outside, by adhesive, suturing, and/or a mechanical fastening device such as a pin. The sheath 140 may be generally tubular in form, with open distal and proximal ends. At or toward its distal end, the sheath may be attached to the plurality of arms. At its proximal end, the sheath may be circumferentially attached to the collet 54, via an o-ring or another fastener. In some embodiments, the outer sheath comprises a mesh interwoven with a secondary material that is conductive. The conductive nature of the mesh may be used to oblate tissue or used in a more diagnostic mode, such as detecting nerve tissue in conjunction with an electromyography (EMG) device. The outer sheath may comprise the same materials as the inner mesh.

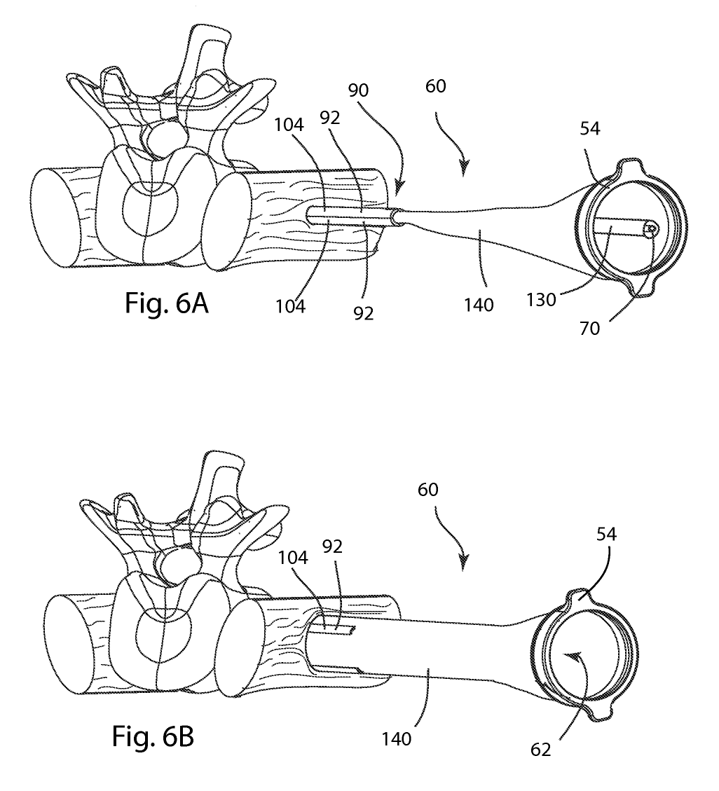

FIG. 6A shows the device 60 in the closed configuration, advanced into a muscle, while FIG. 6B shows the device 60 in the open configuration, dilating the muscle to provide a passageway through the muscle. In FIGS. 6A and 6B, the outer sheath 140 is positioned inside the plurality of arms 90. Referring to FIG. 6A, the device 60 has been partially advanced into the psoas muscle adjacent the spine. The stylus 70 and closed plurality of arms 90 have penetrated the muscle, dissecting the muscle fibers. As the device is introduced, it may be rotated such that the outer surfaces 104 of the arms 92 are placed in a preferred orientation relative to the muscle fibers, so that the fibers may be primarily pushed aside, instead of torn, during dilation. For example, placing the device so that the outer surfaces 104 of the arms 92 are at approximately 45° relative to the longitudinal axis of the muscle fibers may be preferable, as shown in FIGS. 6A and 6B. Fluid is introduced into the bore of the stylus, where it passes through the ports 82 into the balloon 110, inflating the balloon and causing it to expand radially. As the balloon expands, the surrounding inner mesh 130 expands, and the arms 92 are forced radially outward and are radial-laterally displaced from one another, as seen in FIGS. 6B and 4B. The surrounding muscle fibers are dissected and the muscle is dilated, creating a passageway through the muscle to the spine.

After the balloon 110 has been inflated a desired amount, the stylus, balloon and inner mesh may be removed from the device 60, leaving the expanded arms 90 and outer mesh 140 surrounding an open passageway 62. Before or after removal of the stylus, balloon and inner mesh, a rigid cannula may be longitudinally inserted into the passageway 62, inside the arms 90 and outer mesh 140, to further hold the passageway open; the cannula forming an inner wall of the passageway. Instruments, implants and other materials may be passed through the passageway to perform surgical procedures. In the open configuration, the maximum outer diameter of the device 60, measured normal to the longitudinal axis of the stylus and rigid arms such as along line a-a in FIG. 4A, may range from 25 to 40 millimeters. An expansion ratio of the device may be measured as the ratio of maximum outer diameter of the device in the open configuration to the maximum outer diameter of the device in the closed configuration. The expansion ratio of device 60 may range from 2.5 to 8.0. In some embodiments, the expansion ratio may range from 3.0 to 7.5; in other embodiments, the expansion ratio may range from 4.0 to 7.0; while in other embodiments, the expansion ratio may range from 5 to 6.5. In a preferred embodiment, the expansion ratio may be at least 6.0.

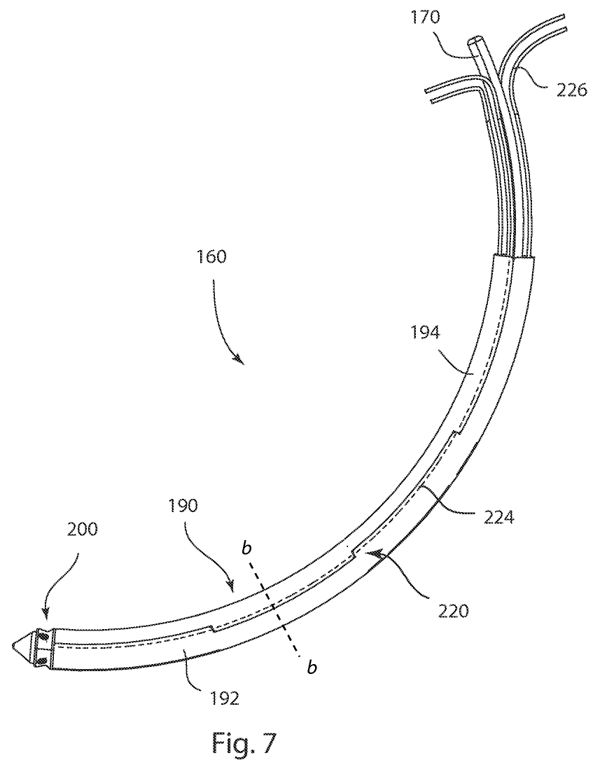

FIGS. 7-10 illustrate another embodiment of an minimally invasive expandable dilation device. Dilation device 160 comprises a curved stylus 170, a plurality of rigid curved arms 190 radially arrayed about the stylus, and two balloons which are circumferentially attached to the stylus. Dilation device 160 may be used in a postero-lateral approach to dilate and form a passageway through the psoas muscle in order to obtain access to an intervertebral space. In other embodiments of the invention, one balloon may be attached to the curved stylus, or a plurality of balloons. In yet other embodiments, at least one cannula may be introduced into the space defined by the arms to expand the arms apart, instead of one or more balloons. Device 170 may be used to create a curved passageway through the psoas muscle, and/or to create a curved passageway through another muscle or set of tissues.

Referring to FIG. 7, the dilation device 160 is shown in a closed configuration, with the plurality of curved arms 190 enclosing and obscuring the balloons. A central longitudinal space is circumferentially surrounded by the arms. The plurality of curved arms 190 comprises four individual curved arms 192, 194, 196, 198. Each arm is releasably secured to the distal end of the stylus via an attachment mechanism 200. Each arm is also releasably secured to the lateral edge of the adjacent arms via lateral engagement features 220. A release wire 240 secures the each arm to its adjacent neighbor by extending through an arm bore 224 which extends the length of the arms, from the proximal end to the distal end. In this embodiment, the arms are not identical to one another but each shaped so that when fitted together the arms form a closed curved cylinder about the curved stylus. For example, arms 194 and 196 may be shorter than arms 192 and 198, and have a slightly smaller radius of curvature than arms 192 and 198. The maximum outer diameter of the device 160 in the closed configuration, measured normal to the longitudinal axes of the stylus and rigid arms, such as along line b-b, may range from 5 to 15 millimeters.

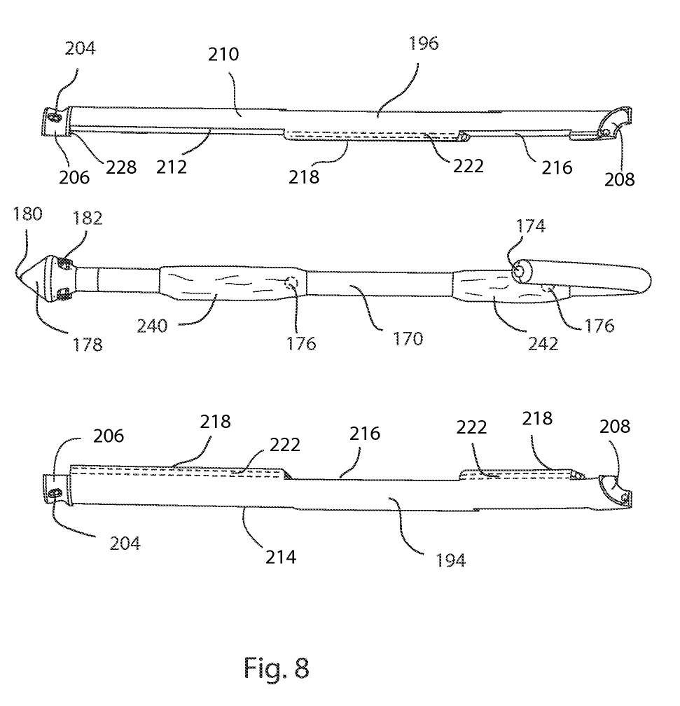

Referring to FIG. 8, a perspective top down view shows the stylus 170 and two arms 194, 196. Mounted on a stylus shaft 172 are two uninflated balloons 240, 242. Each balloon extends longitudinally along a portion of the stylus, and is secured to the stylus at each balloon end. The stylus comprises an inner bore 174 which extends along a length of the stylus, and is in communication with two ports 176. The bore and ports provide a passageway for fluid to inflate the balloons 240, 242. At a distal end 177 of the stylus is a stylus tip 178 which may be formed integrally with, or separately from the stylus. The stylus tip has a point 180 which may be blunt in order to more gently push aside tissues during insertion of the stylus into body tissues and muscles. The stylus tip may have a distal conical surface which also aids in atraumatically parting tissues and muscle fibers. The maximum diameter of the stylus tip may be greater than the shaft of the stylus, as in FIG. 8; in other embodiments the maximum diameter of the stylus tip may be the same or less than the stylus shaft.

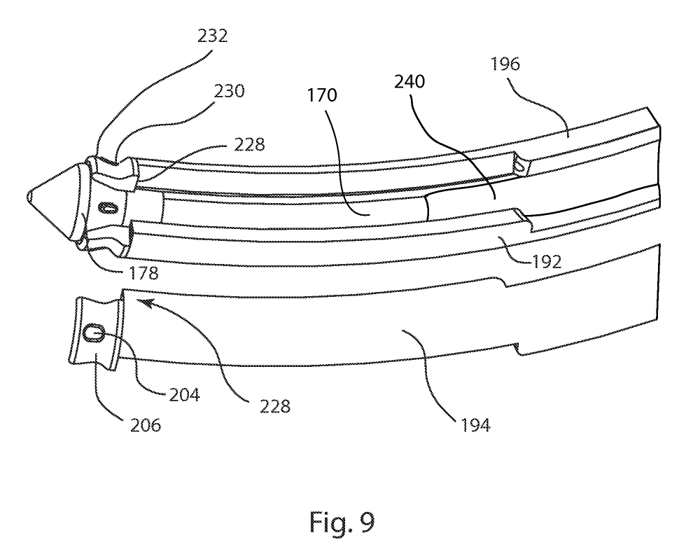

With reference to FIGS. 8 and 9, the stylus tip 178 has four discrete connecting features 182 located adjacent the distal end of the stylus. Each connecting feature 182 is a peg protruding radially from the stylus, each peg having an ovoid or egg shape with one rounded end slightly larger than the other. This shape ensures a close fitting with a complementarily shaped receiving hole 204 on the end of each arm. When the arms are fitted on the pegs as in FIG. 7, the arms are longitudinally aligned with the stylus in a predetermined longitudinal alignment in which the arms cannot move laterally relative to one another until the connection features are detached; e.g., the hole 204 is taken off the peg 182. Other connecting features are contemplated within the scope of the invention, including but not limited to pegs and corresponding holes which are round, oval, rectangular, or multi-sided; or other complementary protrusion and slot combinations. The receiving hole may be open on both ends or may be a recess or cavity with an opening on one side shaped to receive the peg. In an alternative embodiment, the pegs may be located on the arms, and the receiving hole or cavity on the stylus or stylus tip.

Each arm 192, 194, 196, 198 comprises a distal end 206, a proximal end 208, and an arm shaft 210 bounded laterally by a first lateral edge 212 and a second lateral edge 214. Each lateral edge 212, 214 comprises one or more recessed portions 216 which are distributed alternately with projecting portions 218. Thus when two arms are fitted together laterally, the projecting portions 218 on one arm fit into the recessed portions 216 on the adjacent arm. An arm bore section 222 extends longitudinally along each lateral edge, through the entire length of each projecting portion 218. When two arms are fitted together laterally, one continuous arm bore 224 is formed from the alternating arm bore sections 222 which are now axially aligned with one another. A release wire 226, seen in FIG. 7, can be inserted along the length of each arm bore 224 to effectively pin or lace the arms securely together. After advancement of the closed device 160 into the tissues and prior to expansion of the device, the release wire(s) 226 may be withdrawn so that the arms may move apart from one another with the expansion force. Other lateral engagement features are contemplated within the scope of the invention, including but not limited to tongue-in-groove features, corresponding tab and slot features, or press-fit features. Such features may be disengaged by removal of a pin, suture or wire such as release wire 226, or may have a friction fit in which the features are detached from one another by sufficient expansive force provided by expansion of the dilating member.

Referring to FIGS. 8 and 9, details of the arm and stylus distal ends are shown. The distal end 206 of each arm 192, 194, 196, 198 may include an offset 228, in which the distal end is offset from the shaft 210. The offset 228 allows the arms to fit more precisely together when the device 160 is in the closed configuration, and also provides a stop surface for a distal end of the release wire 226. The distal end 206 may also include a waist 230 and an adjacent flared portion 232. Together, the waist 230 and flared portion 232 form a concavely curved area at the distal end of the arm, which may aid in holding back or retaining tissues dissected and pushed aside by the stylus tip 178 during advancement of the device into muscle and other tissues, and may aid in holding back or retaining tissues moved apart during dilation or expansion of the device 160. The flared portions 232 may act as a retainer to prevent tissues from slipping back over the distal ends of the arms once the tissues have been dissected apart from one another. The inner surface of the flared portion 232 may also be shaped to complementarily mate with the outer surface of the stylus tip 178, a portion of which may flare outward.

An alternative embodiment of the dilation device may include a stylus and arms with corresponding connecting features such as the peg/hole system set forth above, but no lateral engagement features on the arms. Another embodiment may include lateral engagement features on two or more arms, but no corresponding connecting features between the arms and the stylus. Yet another embodiment may comprise neither distal connection features nor lateral engagement features. It is appreciated that the contemplated invention may include any combination of the features described herein.

Dilation device 160 may further comprise an inner mesh positioned longitudinally between the balloons and the plurality of arms in the same manner as inner mesh 130 set forth in the previous embodiment. The device may also further comprise an outer sleeve securely attached to the arms and positioned longitudinally either inside or outside the plurality of arms, in the same manner as outer sleeve 140 set forth in the previous embodiment. The mesh and sleeve may comprise the same materials as set forth previously for inner mesh 130.

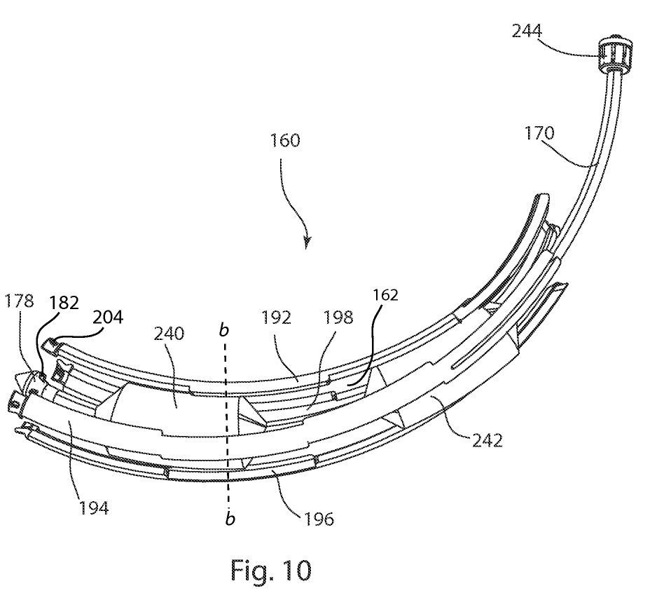

Referring to FIG. 10, dilation device 160 may be expanded by introduction of a fluid into the stylus 170. Prior to expansion, any release wires may be withdrawn from the arm bores. A luer 244 at the distal end of the stylus provides means for introduction of the fluid, such as saline, into the stylus bore. The fluid is forced distally along the stylus and escapes through ports 176, inflating balloons 242 and 240. The proximally located balloon 242 may inflate in advance of the distally located balloon 240, and this may push the arms slightly distally, then radially outward as both balloons inflate. As the arms 192, 194, 196, 198 move radially outward, the arm connection features 204 are disengaged from the stylus connection features 182 by the expansion force provided by the inflation of the balloons. Similarly, the lateral engagement features 216, 218 disengage and the arms may move radial-laterally apart with the expansion of the balloons. After inflation of the balloon has provided sufficient expansion of the device to dilate the surrounding tissue a desired amount, the introduction of fluid may be ceased, and the stylus 170 with the attached balloons 240, 242 may be removed, leaving a passageway through the surrounding tissue. In the open configuration, the maximum outer diameter of the device 160, measured normal to the longitudinal axis of the stylus and rigid arms, such as again along line b-b, may range from 25 to 40 millimeters. The expansion ratio of device 160 may range from 2.5 to 8.0. In some embodiments, the expansion ratio may range from 3.0 to 7.5; in other embodiments, the expansion ratio may range from 4.0 to 7.0; while in other embodiments, the expansion ratio may range from 5 to 6.5. In a preferred embodiment, the expansion ratio may be at least 6.0.

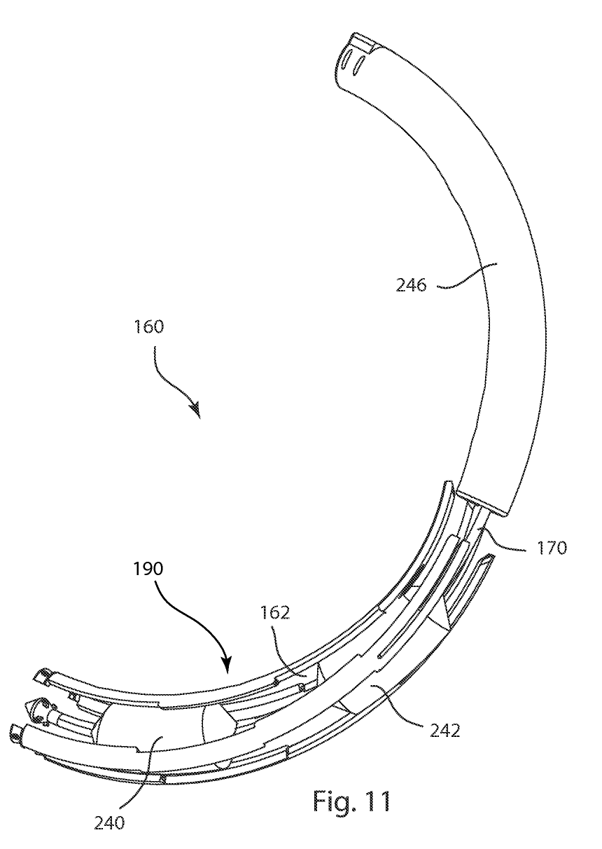

Referring to FIG. 11, a cannula may be inserted between the stylus 170 and the curved arms 190 to keep the device in the open configuration and prevent migration of tissues into the central longitudinal space 162. A cannula such as arcuate cannula 246 may be inserted along a curved path over the stylus 170 before withdrawal of the stylus and balloons from the device 160, as shown in FIG. 11. Alternately, the cannula may be inserted along the insides of the arms after withdrawal of the stylus and balloons. It is appreciated that the cannula is not passed along the outside of the device, which could crush or injure of the adjacent tissues. Instead, the cannula is advanced along the inner sides of the expanded arms, and within the optional outer sleeve. Following insertion of the cannula and withdrawal of the stylus, balloons, and optional inner mesh, instruments, implants and other materials may be passed through the cannula to perform surgical procedures at the end of the passageway formed by the expanded device. The cannula may be docked to a skeletal structure such as a vertebra, and/or to a surgical table support system, to provide stability during surgical procedures.

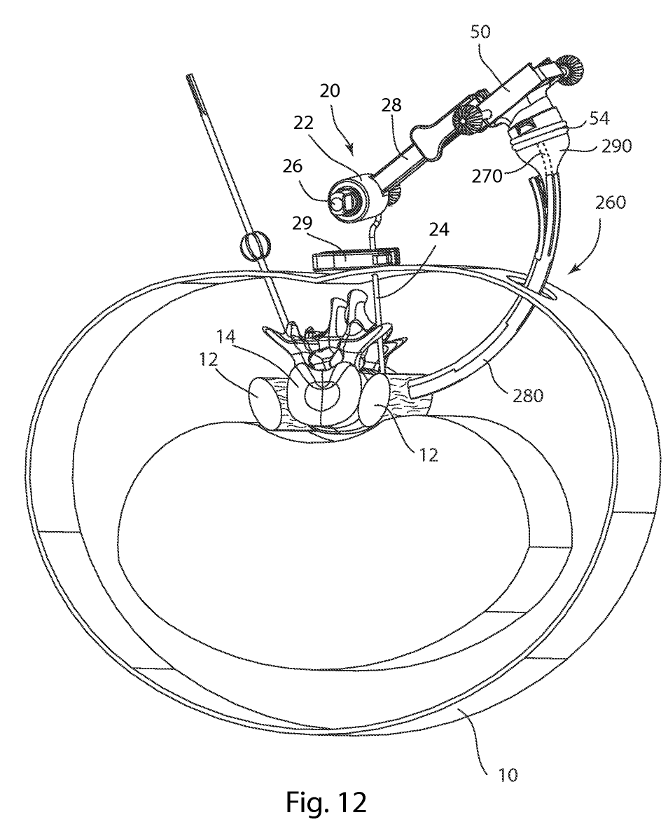

FIGS. 12-14 illustrate a dilation device inserted through an opening in the skin and through a psoas muscle to create a passageway to an intervertebral location, from a postero-lateral approach. The device 260 is transformable from a closed configuration in which each arm is in contacting longitudinal alignment with two other of the arms along their lateral edges, and an open configuration in which the arms are radially displaced from the stylus and laterally displaced from one another. Dilation device 260 comprises a stylus 270, a plurality of arms 280, a tubular sleeve 290, and dilating member which is a cannula 300. In embodiments such as this wherein the dilating member is not a balloon, the entire stylus including the distal tip may be cannulated to allow for flushing of the site, and/or passage of a k-wire.

Referring to FIG. 12, dilation device 260 is shown in the closed configuration, inserted through an incision in the skin 10 and through the psoas muscle 12. The sleeve 290 is secured to the inner surfaces of the arms 280, and at its proximal end, to a collet portion 54 of hub 50. The stylus 270 is also releasably clamped to the hub 50. The hub 50 is secured to a targeting system 20 which is fully described in U.S. patent application Ser. No. 12/357,695, filed on Jan. 22, 2009 and entitled Spinal Access Systems and Methods, the entirety of which is herein incorporated by reference. It is appreciated that hub 50 and/or the targeting system may be secured to other support systems such as surgical table support systems known in the art, to provide stability during device insertion and dilation, and during other surgical procedures. Targeting system 20 comprises a housing 22, a targeting post 24, a micrometer 26 and a swing arm 28. The targeting post 24 may be advanced through the skin and fascia to a desired depth and location adjacent the spine 14, and a targeting depth stop 29 may regulate the depth of the targeting post. The micrometer 26 may be used to finely adjust the position of the targeting post. The offset arm or swing arm 24 connects the housing 22 to the hub 50. The swing arm 24 may be raised or lowered, rotating about the axis of the housing 22, to raise or lower the hub 50 and the associated dilation device 270. In FIG. 12, the swing arm 24 has been lowered sufficiently to guide the device 260 along an arcuate curved path into the psoas muscle.

Toward the proximal end of the plurality of arms 280, each arm comprises a longitudinal slot which extends from the proximal end distally along a portion of the arm. This slot may provide a slight amount of flexibility to the arm proximal ends as the cannula 300 is inserted to initiate transformation of the device 260 from the closed to the open configuration. The slots may also be guides, cooperating with pins or protrusions on the cannula or on a separate guiding ring to guide insertion of the cannula into the device.

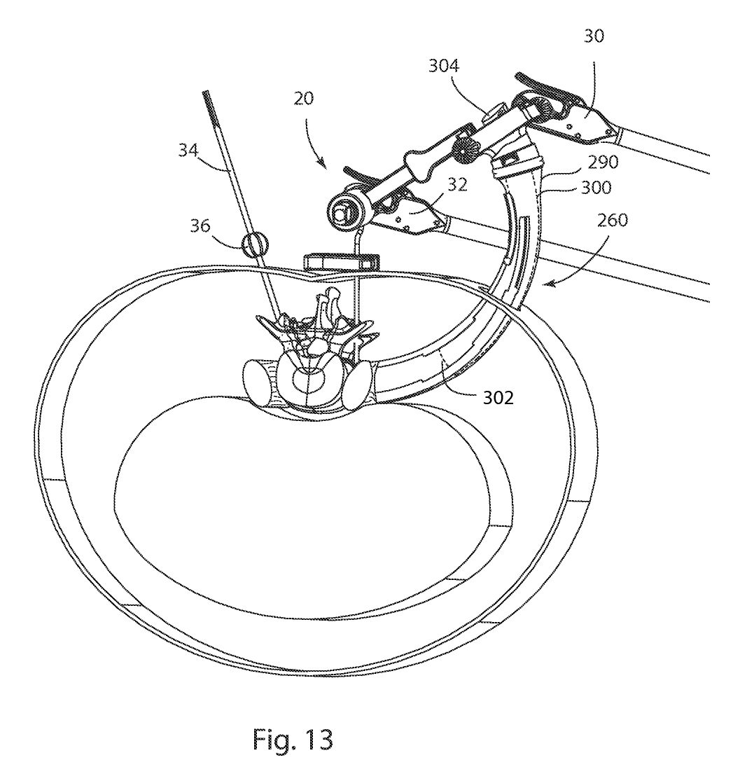

Referring to FIG. 13, the dilation device 260 is shown in the open configuration. The hub 50 and the housing 22 are connected to polyaxially adjustable table mounted clamps 30, 32. A post 34 is anchored in a pedicle, and a slidable clamping sphere 36 is positioned on the post. Optionally, the targeting system may be clamped to the post 34, in place of or in addition to the table mount clamp. The stylus 270 has been withdrawn from the dilation device 260, and a distal end 302 of the cannula 300 has been partially advanced into the system, inside the plurality of arms 280 and the sleeve 290. A proximal end of the cannula 304 is docked to the hub 50. The cannula 300 has a larger diameter than the plurality of arms 280 in the closed configuration. As the cannula 300 is advanced along a curved path inside the space within the closed arms, the arms 280 are forced radial-laterally apart, opening up the attached tubular sleeve 290, and creating a passageway through the tissues and psoas muscle.

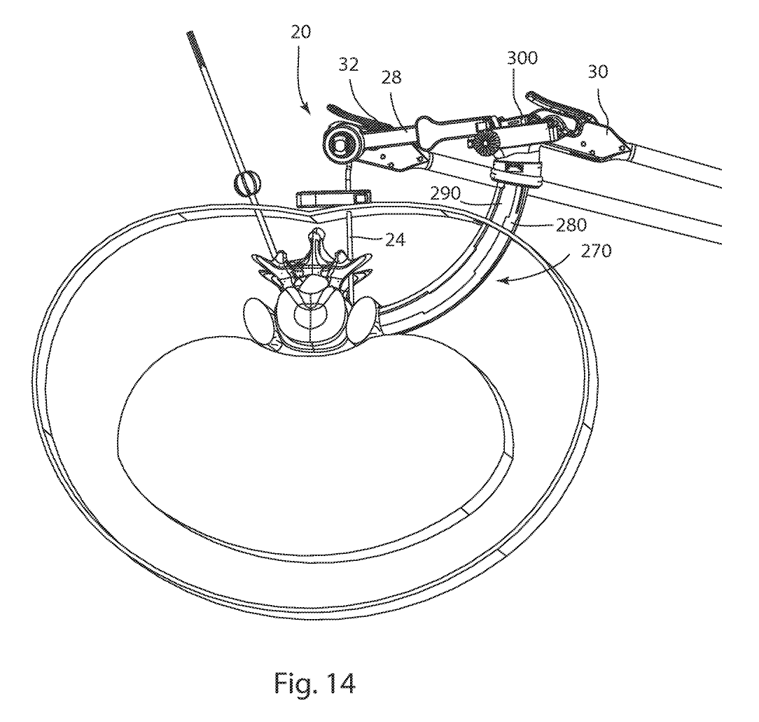

Referring to FIG. 14, the dilation device 260 is shown in the open configuration, and the distal end of the cannula 300 has been fully advanced along the plurality of arms and through the psoas muscle. The swing arm 28 has been partially rotated about the axis of the housing 22, thus lowering the hub and the docked cannula to fully advance the cannula along the curved path, and decreasing the angle between the swing arm 28 and the targeting post 24. The clamp arms 30, 32 have been adjusted to stabilized the device at the fully advanced position. Other instruments, implants, and materials may be passed through the passageway formed by the cannula.

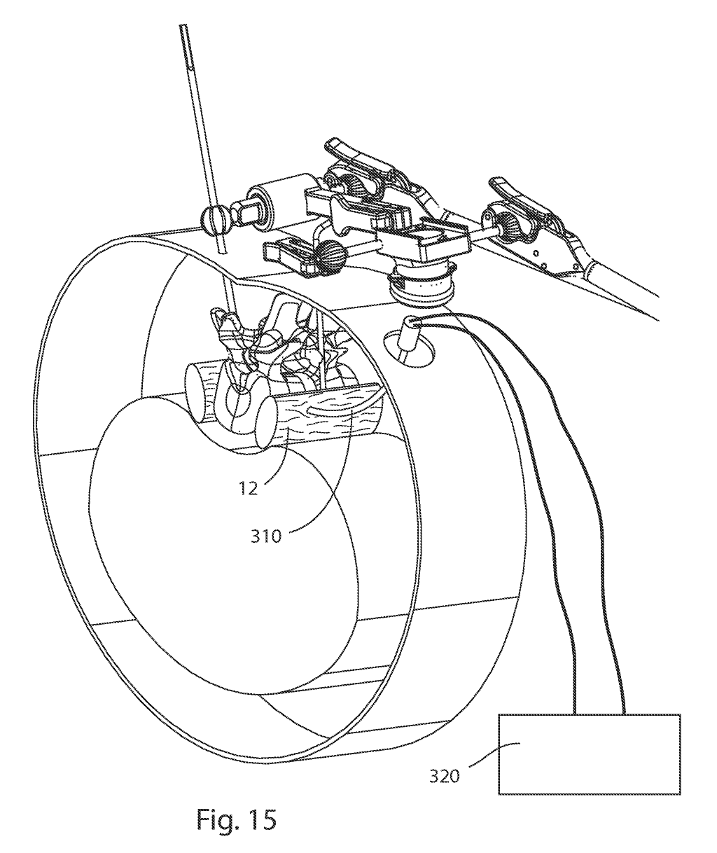

FIG. 15 illustrates an electromyography (EMG) electrode 310 inserted into the psoas muscle. The electrode 310 is connected to a neural monitoring system 320 which can detect the presence of nervous tissue. Prior to insertion of the a dilation device such as devices 60, 160, or 260, the electrode 310 may be advanced into the muscle or tissue to be dilated, and energized, or activated, to detect the presence of a nerve. The electrode may then be deactivated and/or removed, and the dilation device inserted into the muscle or tissue. If a nerve is sensed along a particular path or trajectory, the dilation device may be inserted along a different path or trajectory, in order to avoid the nerve. The electrode and neural monitoring system may be used prior to insertion of the dilation device, after insertion of the device but prior to dilation, and following dilation, to detect and avoid nervous tissue. The electrode may also be activated intermittently during advancement of the dilation device. In alternative embodiments of the device, the rigid arms, if metallic, may be used as the electrode. As well, transmissive tape and/or paint can be applied to the surface of the arms to create a surface capable of transmitting voltage.

One way to view the teachings set forth above is to characterize certain structures as connecting means for placing each arm in a predetermined longitudinal alignment with the stylus. In the various embodiments set forth above the connecting means can be said to be elements 79 and 97 as shown in FIGS. 4A, 5A and 5B, or elements 182 and 204 as shown in FIGS. 8 and 9. Other connecting means are contemplated within the scope of the invention, including but not limited to pegs and corresponding holes which are round, oval, rectangular, or multi-sided; or other complementary protrusion and slot combinations. The receiving hole may be open on both ends or may be a recess or cavity with an opening on one side shaped to receive the peg. In an alternative embodiment, the pegs may be located on the arms, and the receiving hole or cavity on the stylus or stylus tip.

Certain aspects of the teaching set forth above can be characterized as lateral engagement means for placing the arms in contacting longitudinal alignment with one another along their first and second lateral edges. The structure for the lateral engagement means is found in FIGS. 5C and 5D in elements 108, and in FIGS. 7-9 in elements 216, 218, 224 and 226. Other lateral engagement means are contemplated within the scope of the invention, including but not limited to tongue-in-groove features, corresponding tab and slot features, or press-fit features. Such features may be disengaged by removal of a pin, suture or wire such as release wire 226, or may have a friction fit in which the features are detached from one another by sufficient expansive force provided by expansion of the dilating member.

Some aspects of the teaching set forth above can be characterized as a means for dilation. In the various embodiments set forth above the means for dilation can be said to be element 110 in FIGS. 3, 4A, and 4B; elements 240 and 242 in FIGS. 8-11; and element 300 in FIGS. 14 and 15. Other dilation means contemplated in the scope of the invention include expansion instruments such as retractors and other mechanical expanders.

Some aspects of the teaching set forth above can be characterized as a means for circumferentially surrounding at least a portion of the dilating member. In the various embodiments set forth above the means for circumferentially surrounding at least a portion of the dilating member can be said to be elements 130 and 140 in FIGS. 1-4B and FIGS. 6A-6B, element 246 in FIG. 11, and element 290 in FIGS. 12-14.

The present invention may be embodied in other specific forms without departing from its spirit or essential characteristics. It is appreciated that various features of the above-described examples can be mixed and matched to form a variety of other alternatives. For example, the dilating member may comprise a balloon, and/or a cannula. Embodiments may variously include connecting features between the stylus and the plurality of arms, and engagement features between individual arms. It is also appreciated that this system should not be limited creating a passage through a muscle; it may be used to create a passage through any soft tissues. As such, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A device for forming an open passage through tissue, the device comprising:
a rigid stylus comprising: a distal end, a proximal end, a stylus shaft extending from the distal end to the proximal end, and a plurality of connecting features formed on the stylus adjacent the distal end;
a plurality of rigid arms, each rigid arm comprising: a distal end, a proximal end, an arm shaft extending from the distal end to the proximal end, and a connecting feature formed on the arm adjacent the distal end, the arm connecting feature shaped to engage one of the stylus connecting features to place the arm in a predetermined longitudinal alignment with the stylus when the connecting features are engaged;
each arm shaft having a first lateral edge and a second lateral edge opposite the first lateral edge, the first lateral edge having a first lateral engagement feature, the second lateral edge having a second lateral engagement feature; and
a dilating member positioned between the rigid stylus and the plurality of rigid arms;
wherein in response to actuation of the dilating member, the device is transformable between a closed configuration in which each arm connecting feature is engaged with one of the stylus connecting features and each arm is in contacting longitudinal alignment with two other of the arms along their lateral edges, and an open configuration in which the arm connecting features are disengaged from the stylus connecting features and the arms are radially displaced from the stylus and radial-laterally displaced from one another.

2. The device of claim 1, wherein the first lateral engagement feature of each arm is shaped to cooperate with the second lateral engagement feature of an adjacent one of the arms to place the arms in contacting longitudinal alignment with one another along their first and second lateral edges.

3. The device of claim 1, further comprising:
an expandable sleeve, the sleeve generally tubular and having a distal end, a proximal end, and a sleeve body extending from the distal end to the proximal end, the sleeve open at at least the proximal end, the sleeve body circumferentially surrounding at least a portion of the dilating member.

4. The device of claim 1, wherein the dilating member comprises a balloon, the balloon attached to the stylus shaft.

5. The device of claim 1, wherein the dilating member comprises a rigid cannula removably insertable between the stylus and the plurality of rigid arms.

6. The device of claim 1, wherein when in both the closed and open configurations, the entire device comprises a substantially cylindrical shape.

7. The device of claim 1, wherein the device comprises an outer diameter expansion ratio of at least 4.0 when transformed from the closed configuration to the open configuration.

8. The device of claim 1, wherein each of the stylus shaft, the dilating member and each of the rigid arm shafts is curved along its entire length.

9. The device of claim 1, wherein the stylus connecting features project outward from the stylus shaft in a regular radial distribution.

10. The device of claim 1, wherein the plurality of rigid arms forms a closed radial array about the stylus when the device is in the closed configuration.

11. The device of claim 10, wherein the distal end of each rigid arm is curved such that the distal end flares radially outward relative to the stylus when the rigid arms are radially arrayed about the stylus.

12. A method for forming a passage through tissue, the method comprising:
inserting a dilation device into the tissue, the dilation device comprising:
a rigid stylus having a stylus shaft extending from a distal end to a proximal end, and a plurality of connecting features formed on the stylus adjacent the distal end;
a plurality of rigid arms, each rigid arm having a connecting feature formed on the arm adjacent a distal end of the arm, the arm connecting feature in engagement with one of the stylus connecting features such that the arm is in a predetermined longitudinal alignment with the stylus, each rigid arm further comprising a first lateral edge and a second lateral edge opposite the first lateral edge, each rigid arm immediately adjacent two other rigid arms; and
a dilating member positioned between the rigid stylus and the plurality of rigid arms;
radially expanding the dilating member, wherein upon the radial expansion of the dilating member, the dilating member provides radially projecting force which pushes radially outward on the plurality of arms, transforming the device from a closed configuration to an open configuration;
disengaging the arm connecting features from the stylus connecting features;
radially displacing each arm from the stylus;
radial-laterally displacing each arm from the immediately adjacent other arms; and
withdrawing the stylus and the dilating member from the dilation device and the tissue, thereby leaving an open passage through the dilation device and the tissue.

13. The method of claim 12, wherein when the device is in the closed configuration, a first lateral engagement feature of each arm is in cooperation with a second lateral engagement feature with another of the arms such that each arm is in contacting longitudinal alignment with the two immediately adjacent other arms along their first and second lateral edges.

14. The method of claim 12, wherein transforming the device from the closed configuration to the open configuration comprises expanding the device with an outer diameter expansion ratio of at least 4.0.

15. The method of claim 12, further comprising advancing a cannula distally between the rigid stylus and the plurality of arms such that the cannula circumferentially surrounds the stylus and the plurality of arms are radially arrayed about the cannula.

16. The method of claim 12, further comprising passing at least a portion of a surgical instrument through the open passage.

17. The method of claim 12, wherein the dilation device further comprises an expandable sleeve circumferentially surrounding at least a portion of the dilation member, and wherein radially expanding the dilation member radially expands the expandable sleeve.

18. The method of claim 12, wherein the dilation device is curved along its entire length between a proximal end and a distal end, and wherein inserting the dilation device into the tissue further comprises inserting the dilation device along a curved path.

19. The method of claim 12, wherein the dilation device comprises a balloon member, wherein radially expanding the dilation device further comprises inflating the balloon member.

20. A system for forming an open passage through tissue, the system comprising:
a dilation device insertable into the tissue, the device comprising:
a radially expandable array, the array comprising a plurality of independent rigid arms positioned regularly about and defining a central longitudinal space, each arm having a distal end, a proximal end, and an arm shaft extending from the distal end to the proximal end;
a cylindrical balloon member positioned in the central longitudinal space, the balloon member inflatable to radially expand upon introduction of a fluid into a lumen of the balloon member; and
an expandable sleeve, the sleeve generally tubular and having a distal end, a proximal end, and a sleeve body extending from the distal end to the proximal end, the sleeve body circumferentially attached to least a portion of the radially expandable array; and
a nerve stimulating electrode insertable into the tissue to detect the location of nerves within the tissue;
wherein upon introduction of the device into the tissue following insertion of the nerve stimulating electrode and detection of nerves, and upon inflation of the balloon member, expansion of the balloon radially expands the array and the expandable sleeve to form an open passage through the tissue while avoiding contact with nerves within the tissue.

21. The system of claim 20, further comprising a rigid stylus comprising a stylus shaft, wherein the balloon member is attached to the rigid stylus such that the stylus shaft passes through the central lumen of the balloon member.

22. The device of claim 21, wherein each of the stylus shaft, the balloon member and each of the rigid arm shafts is curved along its entire length.

23. The device of claim 21, wherein the stylus shaft comprises a plurality of stylus connection features, and wherein each rigid arm comprises an arm connecting feature shaped to engage a stylus connecting feature such that the arm is in a predetermined longitudinal alignment with the stylus.

24. The device of claim 20, wherein the dilation device comprises an outer diameter expansion ratio of at least 4.0 when radially expanded.

25. The device of claim 20, wherein each rigid arm further comprises a first lateral edge having a first lateral engagement feature, a second lateral edge having a second lateral engagement feature, the first lateral engagement feature of each arm shaped to cooperate with the second lateral engagement feature of two other of the arms.

26. A method for forming an open passage through a tissue, the method comprising:
inserting a nerve stimulating electrode into the tissue;
activating the nerve stimulating electrode to sense the presence of a nerve;
advancing a dilation device into the tissue, the dilation device separate from the nerve stimulating electrode, the dilation device comprising:
a radially expandable array, the array comprising a plurality of independent rigid arms positioned regularly about and defining a central longitudinal space, each arm having a distal end, a proximal end, and an arm shaft extending from the distal end to the proximal end;
a balloon member positioned in the central longitudinal space; and
an expandable sleeve, the sleeve circumferentially surrounding at least a portion of the radially expandable array;
inflating the balloon member to radially expand the expandable array and the surrounding sleeve to form a passage through the tissue.

27. The method of claim 26, further comprising removing the balloon member from the central longitudinal space to provide an open channel through the central longitudinal space.

28. The method of claim 27, wherein the device further comprises a stylus upon which the balloon member is mounted, the method further comprising:
removing the stylus with the balloon member from the central longitudinal space to provide the open channel through the central longitudinal space.

29. The method of claim 26, further comprising inserting a rigid cannula into the central longitudinal space to prevent retraction of the radially expandable array into the central longitudinal space.

30. The method of claim 26, further comprising further comprising passing at least a portion of a surgical instrument through the open passage.

31. The method of claim 26, further comprising:
deactivating the nerve stimulating electrode after activating the nerve stimulating electrode and before advancing the dilation device into the tissue;
ceasing advancing the dilation device into the tissue to avoid the nerve;
reactivating the nerve stimulating electrode to sense the presence of a nerve;
deactivating the nerve stimulating electrode; and
readvancing the dilation device into the tissue along a path to avoid the nerve.

32. The method of claim 26, wherein the tissue is a muscle, the method further comprising:
inflating the balloon member to radially expand the expandable array and the surrounding sleeve to separate the fibers of the muscle and form a passage through the muscle.

33. A device for forming an open passage through tissue, the device comprising:
a rigid stylus comprising: a distal end, a proximal end, a stylus shaft extending from the distal end to the proximal end;
a plurality of rigid arms, each rigid arm comprising: a distal end, a proximal end, an arm shaft extending from the distal end to the proximal end, each arm shaft having a first lateral edge and a second lateral edge opposite the first lateral edge;
connecting means for placing each arm in a predetermined longitudinal alignment with the stylus;
lateral engagement means for placing the arms in contacting longitudinal alignment with one another along their first and second lateral edges; and
dilating means for transforming the device between a closed configuration in which each arm is in a predetermined longitudinal alignment with the stylus and each arm is in contacting longitudinal alignment with two other of the arms along their lateral edges, and an open configuration in which the arms are radially displaced from the stylus and radial-laterally displaced from one another.

34. The device of claim 33, further comprising:
means for circumferentially surrounding at least a portion of the dilating member.

35. The device of claim 33, wherein the dilating means comprises a balloon, the balloon attached to the stylus shaft.

36. The device of claim 33, wherein the dilating means comprises a rigid cannula removably insertable between the stylus and the plurality of rigid arms.

37. The device of claim 33, wherein the device comprises an outer diameter expansion ratio of at least 4.0 when transformed from the closed configuration to the open configuration.

38. The device of claim 33, wherein each of the stylus shaft, the dilating means and each of the rigid arm shafts is curved along its entire length.

39. The device of claim 33, wherein the connecting means comprises a plurality of stylus connecting features which project outward from the stylus shaft in a regular radial distribution.

40. The device of claim 33, wherein the plurality of rigid arms forms a closed radial array about the stylus when the device is in the closed configuration.

41. The device of claim 33, wherein the distal end of each rigid arm is curved such that the distal end flares radially outward relative to the stylus when the rigid arms are radially arrayed about the stylus.
